(19) United States
(12) Patent Application Publication
(10) Pub. No.: US 2025/0258108 A1
(43) Pub. Date: Aug. 14, 2025

(54) INSPECTION DEVICES AND INSPECTION METHODS

(71) Applicant: TOPPAN HOLDINGS INC., Tokyo (JP)

(72) Inventors: Ryoichi FUKAI, Taito-ku (JP); Atsushi SHIGA, Taito-ku (JP); Fumitaka UETO, Taito-ku (JP); Shinya TAKAHASHI, Taito-ku (JP); Maiko KATO, Taito-ku (JP)

(73) Assignee: TOPPAN HOLDINGS INC., Tokyo (JP)

(21) Appl. No.: 19/190,996

(22) Filed: Apr. 28, 2025

Related U.S. Application Data
(63) Continuation of application No. PCT/JP2023/039317, filed on Oct. 31, 2023.

(30) Foreign Application Priority Data
Oct. 31, 2022 (JP) 2022-174411

(51) Int. Cl. G01N 21/88; G01N 9/02; G01N 21/84; G01N 33/02

(52) U.S. Cl. CPC G01N 21/8851; G01N 9/02; G01N 33/025; G01N 2021/8466

(57) ABSTRACT

An inspection system includes a base on which an object to be inspected is placed, a drive device that rotates the base on which the object to be inspected is placed, and an imaging device that images the object to be inspected.

### CROSS-REFERENCE TO RELATED APPLICATIONS

The present application is a continuation of and claims the benefit of priority to International Application No. PCT/JP2023/039317, filed Oct. 31, 2023, which is based upon and claims the benefit of priority to Japanese Application No. 2022-174411, filed Oct. 31, 2022. The entire contents of these applications are incorporated herein by reference.

### BACKGROUND OF THE INVENTION

### Field of the Invention

The present invention relates to inspection devices and inspection methods.

### Description of Background Art

In recent years, against the backdrop of labor shortages in agriculture, automation of inspection and sorting of fruit and vegetables is being promoted. Current fruit sorting criteria include criteria that are easy to measure and quantify (weight and length), as well as criteria that evaluate internal quality difficult to measure (sugar content and acidity), and criteria that evaluate external quality whose evaluation indicators are complex (rot and blemishes), for example, JPH1159877 A, JP 2011-240257 A, JP 2005-046794 A, JP 2019-211456 A, JP 2002-139433 A, JPH07128321 A, JPH08262006 A, JPH0979965 A and JP 2021-135275 A. The entire contents of these publications are incorporated herein by reference.

### SUMMARY OF THE INVENTION

According to one aspect of the present invention, an inspection device includes a base on which an object to be inspected is placed, a drive device that rotates the base on which the object to be inspected is placed, and an imaging device that images the object to be inspected.

According to another aspect of the present invention, an inspection device includes a base on which an object to be inspected is placed, a structure bent obliquely upward from the base, and imaging devices that are supported by the structure and image the object to be inspected in different directions.

According to yet another aspect of the present invention, an inspection device includes a base on which an object to be inspected is placed, a case that supports the base in a non-rotatable manner, a drive device that is positioned in the case and rotates the case, and a support attached to the case and supporting an imaging device that images the object to be inspected.

According to still another aspect of the present invention, an inspection method includes causing a drive device to rotate a base on which an object to be inspected is placed, controlling an imaging device such that the imaging device images the object to be inspected while rotating the object to be inspected and acquiring multiple images of the object to be inspected from the imaging device, and performing inspections of the object to be inspected based on the images of the object to be inspected.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a diagram illustrating an example configuration of an inspection system according to an embodiment;

FIG. 2 is a diagram illustrating an appearance of an inspection device included in the inspection system;

FIG. 3 is a diagram illustrating a modified example of a rotation table;

FIG. 4 is a diagram illustrating example components constituting an inspection device;

FIG. 5 is a diagram illustrating an example hardware configuration of an information processing device;

FIG. 6 is a diagram illustrating an example functional configuration of an inspection program executed by a processor;

FIG. 7 is a flowchart showing an overview of the operation of the inspection system;

FIGS. 8A and 8B are diagrams for explaining a basic method for determining “volume” of an object to be inspected;

FIG. 9 is a diagram for explaining a basic method for determining “deformity” of an object to be inspected;

FIG. 10 is a diagram for explaining the “size” of an object to be inspected;

FIG. 11 is a diagram for explaining the “deformity” of an object to be inspected;

FIG. 12 is a diagram for explaining the “volume” of an object to be inspected;

FIG. 13 is a diagram for explaining the “volume” of an object to be inspected;

FIG. 14 is a diagram for explaining the “volume” of an object to be inspected;

FIG. 15 is a diagram showing an example of results of actual verification performed on “deformity”;

FIG. 16 is a diagram showing correlation between “specific gravity” and “sugar content”;

FIG. 17 is a diagram showing an example of configuration in which multiple inspection devices 1 are used;

FIG. 18 is a diagram illustrating an appearance of an inspection device according to a first example of Modified Example 1;

FIG. 19 is a diagram illustrating an appearance of an inspection device according to a second example of Modified Example 1;

FIG. 20 is a block diagram illustrating a configuration of an inspection device according to Modified Example 1;

FIG. 21 is a diagram illustrating an internal configuration of a case 10 in Modified Example 2; and

FIG. 22 is a block diagram illustrating a configuration of an inspection device according to Modified Example 2.

### DETAILED DESCRIPTION OF THE EMBODIMENTS

Embodiments will now be described with reference to the accompanying drawings, wherein like reference numerals designate corresponding or identical elements throughout the various drawings.

### Configuration

The following description will be given of a configuration of an inspection system according to an embodiment of the present invention.

### Overall System

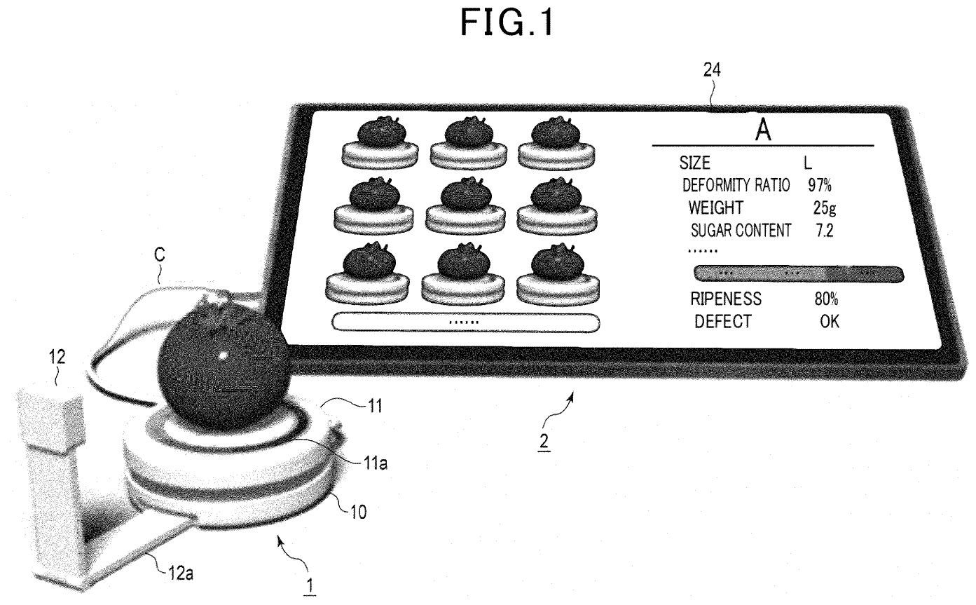

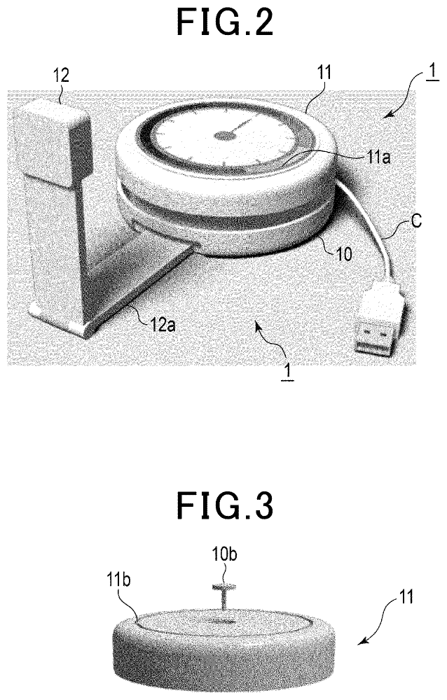

FIG. 1 is a diagram illustrating an example configuration of an inspection system according to an embodiment. FIG. 2 is a diagram illustrating an appearance of an inspection device included in the inspection system.

An inspection system shown in FIG. 1 includes an inspection device 1 and an information processing device 2. The inspection device 1 is a dedicated device for inspecting (or sorting) fruit and vegetables and the like. The information processing device 2 corresponds to a computer (such as a tablet-type information terminal).

As shown in FIGS. 1 and 2, the inspection device 1 includes a case 10 that houses equipment therein, a base 11 (hereinafter, referred to as a “rotation table 11”) on which an object to be inspected such as a fruit or vegetable is placed, a color reference sheet 11a used for color matching during imaging, an imaging device 12 that captures images of the object to be inspected placed on the rotation table 11, a support 12a that supports the imaging device 12, a cable C with a connector, and the like.

If it is difficult to place an object to be inspected on the rotation table 11 in a desired posture, an appropriate tool may be used to hold the desired posture. Although FIG. 1 shows an example in which an object to be inspected is a tomato, the object to be inspected is not limited to tomatoes and may be various types of fruit and vegetables such as onions, apples, tangerines, and the like. Further, the object to be inspected may be any object other than fruit or vegetables.

The color reference sheet 11a is disposed on an upper surface of the rotation table 11 on which an object to be inspected is placed, and is used for calibrating the imaging device 12. The color reference sheet 11a can be used as a guide for color matching and imaging at regular intervals.

The outer materials of the case 10, the rotation table 11, and the like of the inspection device 1 may be metal, but may also be resin. When resin is used, it can be easily and inexpensively manufactured using a 3D printer or the like. The use of resin can reduce the overall weight of the inspection device 1. If there is concern that the stability of the inspection device 1 may be reduced due to the lighter weight, a weight can be placed at the bottom of the case 10 to prevent it from falling over and improve stability.

A small camera module (not shown) is embedded inside the imaging device 12.

The support 12a, which supports the imaging device 12, can be pulled out from the case 10 by the required length. The support 12a may be marked with a scale so that the length of the support 12a pulled out from the case 10 can be determined.

Although not shown in FIGS. 1 and 2, the inspection device 1 contains various types of equipment such as a drive device including a motor and gears that rotate the rotation table 11, and a load cell (weight measuring device) that measures the weight of the object to be inspected placed on the rotation table 11. The details of these devices will be described later.

As shown in FIG. 1, the inspection device 1 is connected to the information processing device 2 via the cable C. The end of the cable C has a connector with an interface function, such as USB 2.0. With this interface function, the information processing device 2 can supply electric power to the inspection device 1, instruct the inspection device 1 to measure the weight of the object to be inspected placed on the rotation table 11, instruct the inspection device 1 to control the rotational movement of the rotation table 11, control imaging by the imaging device 12, control the indicator described later, acquire multiple images generated by the imaging device 12, and obtain weight data of the object to be inspected.

The information processing device 2 uses multiple images and weight data of the object to be inspected obtained from the inspection device 1 to perform inspection (including sorting processing) of various inspection criteria on the object to be inspected, and can display the inspection results of inspection criteria and the final sorting results on the display unit 24.

In the example described herein, the information processing device 2 with the display unit 24 is connected to the inspection device 1 via the cable C, but the configuration is not limited thereto. For example, a small-sized information processing device having the same function as that of the information processing device 2 may be mounted in the inspection device 1, and a display device corresponding to the display unit 24 may be connected to the inspection device 1 instead of connecting the information processing device 2. Further, for example, if a single-board computer is built into the inspection device 1 itself, it can be used as an edge device without the need to connect it to the information processing device 2 via the cable C.

The information processing device 2 is connected to the inspection device 1 via the cable C and controls the above-mentioned drive device via an I/F circuit unit 13 to rotate the rotation table 11 on which the object to be inspected is placed at a constant speed. During the constant speed rotation of the object to be inspected, the information processing device 2 controls the imaging device 12 to capture images of the object to be inspected at regular time intervals. The information processing device 2 has functions of acquiring multiple images of the object to be inspected from the imaging device 12, performing various inspections on the object to be inspected by processing various information based on the acquired plurality of images of the object to be inspected, and displaying the inspection results on the display unit 24.

In the inspection, the information processing device 2 performs inspections for various inspection criteria using the images of the object to be inspected acquired by imaging and the weight data of the object to be inspected. The various inspection criteria herein are “size,” “deformity,” “volume,” “density,” “sugar content,” and “ripeness.”

For example, the information processing device 2 has a function of determining the size of the object to be inspected based on predetermined information obtained from each of the images. The information processing device 2 also has a function of determining the degree of deformity of the object to be inspected based on predetermined information obtained from each of the images. The information processing device 2 also has a function of determining the volume of the object to be inspected based on the sum of predetermined information obtained from each of the images. The information processing device 2 also has a function of determining the density of the object to be inspected based on the volume and weight of the object to be inspected. The information processing device 2 also has a function of determining the sugar content of the object to be inspected based on the density of the object to be inspected. The information processing device 2 also has a function of determining the ripeness of the object to be inspected based on predetermined information obtained from each of the images. The specific information processing and calculation methods for each of the functions will be described later.

The example shown in FIGS. 1 and 2 is merely an example, and the invention is not limited thereto. For example, the color reference sheet 11a may be omitted if it is not necessary. Further, for example, a light emitting unit of the display may be provided at the location of the color reference sheet 11a. Further, for example, the shapes of the imaging device 12 and the support 12a may be modified as appropriate. An example of the rotation table 11 modified according to these viewpoints is shown in FIG. 3.

FIG. 3 is a diagram illustrating a modified example of the rotating table 11.

In the example of FIG. 3, the above-mentioned color reference sheet 11a is not provided on the rotation table 11, but a light emitting unit 11b is provided. The light emitting unit 11b is a portion that emits light of an indicator 16, which will be described later. Further, a fixation member 10b is mounted on the rotation table 11. The fixation member 10b fixes the rotation table 11 to the case in a rotatable manner.

### Configuration of Inspection Device

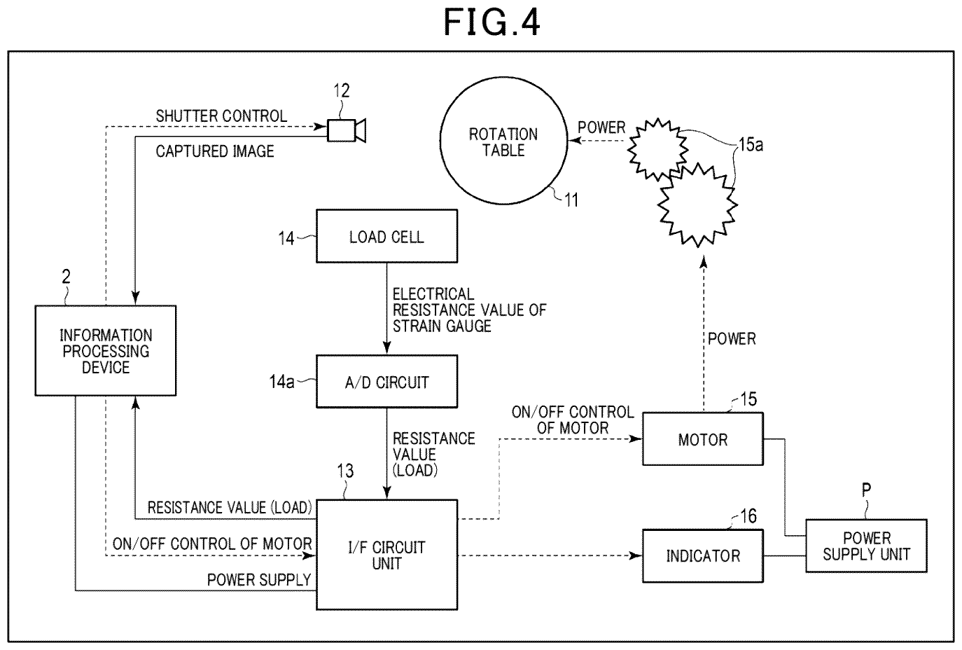

FIG. 4 is a diagram illustrating example components constituting the inspection device 1.

### Components

In addition to the case 10, the rotation table 11, the imaging device 12, the support 12a and the cable C described above, the inspection device 1 includes an interface circuit unit 13, a load cell (weight measuring device) 14, an A/D circuit 14a, a power supply unit (including a battery) P, a motor 15, gears 15a, an indicator 16, and the like as main components.

The case 10 described above contains an interface circuit unit (general interface board) 13 provided with an A/D circuit 14a, a load cell 14 and a power supply unit P. A tray (not shown) disposed on the case 10 is provided with a drive device including the motor 15 and the gears 15a for rotating the rotation table 11. Further, the indicator 16 is disposed on the rear surface of the side wall of the rotation table 11. The indicator 16 includes light emitting diodes (LEDs) of various colors that operate inside the inspection device 1 and changes the color of the LEDs depending on the processing status of the inspection.

The imaging device 12 and the information processing device 2 are electrically connected to each other via the support 12a, the interface circuit unit 13 and the cable C. The results measured by the load cell 14 are transmitted to the interface circuit unit 13 through the A/D circuit 14a. The power supply unit P includes a battery as an auxiliary power supply in case the electric power supplied from the information processing device 2 alone is insufficient. The battery may be, for example, a dry cell or a secondary battery.

The inspection device 1 may perform, for example, the following processing.

Electric power from the power supply unit P rotates the motor 15, which moves the gears 15a on the rear side of the rotation table 11, causing the rotation table 11 to rotate at a constant speed. Further, electric power from the power supply unit P causes the indicator 16 to illuminate.

The information processing device 2 controls the image capturing timing of the imaging device 12 in accordance with the rotation of the rotation table 11 to capture images of the object to be inspected at regular time intervals. Multiple images obtained from the imaging are transmitted to the information processing device 2.

When a fruit or vegetable or the like is placed on the rotation table 11, the load cell 14 is subjected to pressure, and an electrical signal generated from the load cell 14 due to the pressure is A/D converted (analog/digital conversion) by the A/D circuit 14a, and converted into a numerical value to measure the mass of the object to be inspected. The information on the measured mass of the object to be inspected is transmitted to the information processing device 2.

### Rotation Mechanism

The inspection device 1 contains the small motor 15 and the gears 15a, which are arranged in mesh with each other. The gears 15a are also located on the rear side of the rotation table 11, and the gears mesh with each other to form a rotation mechanism around the rotation axis.

Since it is anticipated that the electric power supplied from the cable C alone may not be sufficient to drive the motor 15, power supply from a battery such as an AA battery may be used. In addition, the electric power to drive the motor 15 may be substituted by a combination of a battery having a power generating function, such as a solar cell, and a secondary battery.

The speed of the rotation table 11 can be controlled by adjusting the output of the motor 15 and the shape of the gears 15a. Further, the on/off control of the rotation operation of the rotation table 11 may be performed according to the comparison results between the pressure (weight) sensed by the load cell 14, which will be described later, and a predetermined threshold, or may be performed according to an instruction from the information processing device 2.

### Imaging Mechanism

A small camera module embedded in the imaging device 12 captures images of the object to be inspected at regular intervals according to the control from the information processing device 2.

Imaging is performed by placing the object to be inspected at the center of the rotation table 11, and rotating it at a constant speed. Imaging is performed multiple times at regular intervals from the start of imaging until the object to be inspected makes one turn. Alternatively, it is possible to adopt a method of capturing a video and then extracting images later. In imaging, any number of images can be obtained by adjusting the rotation speed of the rotation table 11 and the shutter timing of the imaging device 12.

The lens may be a wide-angle lens in order to ensure a sufficient imaging field of view. Further, since the support 12a that supports the imaging device 12 is configured to be pulled out from the case 10, the distance between the imaging device 12 and the object to be inspected can be adjusted by pulling out the required length according to the object to be inspected, whereby the range of the field of view of the imaging device 12 can be adjusted.

However, in order to avoid a decrease in accuracy of the inspection, it is desired not to unnecessarily change the imaging conditions such as the distance between the imaging device 12 and the object to be inspected, and the length pulled out from the case 10 may be determined in advance. The distance to be pulled out from the case 10 may be presented to the user from the information processing device 2 via the display unit 24.

### Weight Measuring Mechanism

Weight Measurement is performed using the load cell 14. The load cell 14 is a sensor configured to detect force. The load cell 14 as a sensor converts physical force into an electrical signal, and outputs an electronic signal. The electronic signal can be converted into a weight value using a computer or the like.

Here, it is not necessarily required to measure the weight with the load cell 14 since it is sufficient to introduce a weight measuring mechanism into the rotation table 11. Other possible measurement methods include spring scales, electromagnetic scales and tuning fork vibration scales. In the present embodiment, the load cell 14 is adopted as a device suitable for miniaturizing the weight measuring mechanism provided in the inspection device 1.

The weight measuring mechanism of the load cell 14 utilizes the fact that the amount of electricity output varies depending on the amount of deflection of the strain generating body. For example, one side of a strain generating body made of aluminum is fixed, and a sample is placed on the other side. Then, the weight of the sample causes the strain generating body to deflect, which causes a strain gauge attached to the strain generating body to expand and contract depending on the amount of deflection, varying the amount of electricity output (more specifically, the resistance value). Then, the mass is calculated from the amount of electricity.

### Control Mechanism of Inspection Device

Next, the correlation between the control mechanisms included in the inspection device 1 will be described.

The power supply unit P supplies electric power to the motor 15 and the indicator 16. Specifically, the power supply unit P is a power supply provided with a battery such as a dry cell, and when the electric power supplied from the cable C alone is insufficient, the electric power from the battery is supplied to the indicator 16, the motor 15, and the like.

The load cell 14 transmits the resistance value of the strain gauge to the A/D circuit 14a according to the weight of the object to be inspected placed thereon.

The A/D circuit 14a outputs the resistance value (value corresponding to the load) after the A/D conversion of the resistance value of the strain gauge. Specifically, the A/D circuit 14a converts the electrical resistance value (analog data) of the strain gauge obtained from the load cell 14 into a load (digital data), and transmits this to the interface circuit unit 13 as weight data.

The interface circuit unit 13 mainly integrates various sensor information and the like, and exchanges information with the information processing device 2. For example, the interface circuit unit 13 operates by receiving electric power from the information processing device 2. Further, the interface circuit unit 13 controls the A/D circuit 14a, the motor 15 and the indicator 16.

The interface circuit unit 13 as a general interface board includes the above-mentioned A/D circuit 14a, as well as a motor drive control unit and an indicator drive control unit (not shown). The motor drive control unit controls the motor 15 according to the information on the control of the motor 15 from the information processing device 2. The interlocking of the gears driven by the movement of the motor 15 provides force to rotate the rotation table 11. The indicator drive control unit controls the indicator 16 according to the information on the control of the indicator 16 from the information processing device 2.

The motor 15 transmits the force to the rotation table 11 via the gears 15a, and starts/stops the rotation of the rotation table 11 according to the on/off control of the interface circuit unit 13.

The information processing device 2 supplies power to the interface circuit unit 13, acquires information on the weight of the object to be inspected from the resistance value (value corresponding to the load) after the A/D conversion supplied from the interface circuit unit 13, controls the shutter of the imaging device 12, acquires captured images from the imaging device 12, and performs various inspections.

### Configuration of Information Processing Device

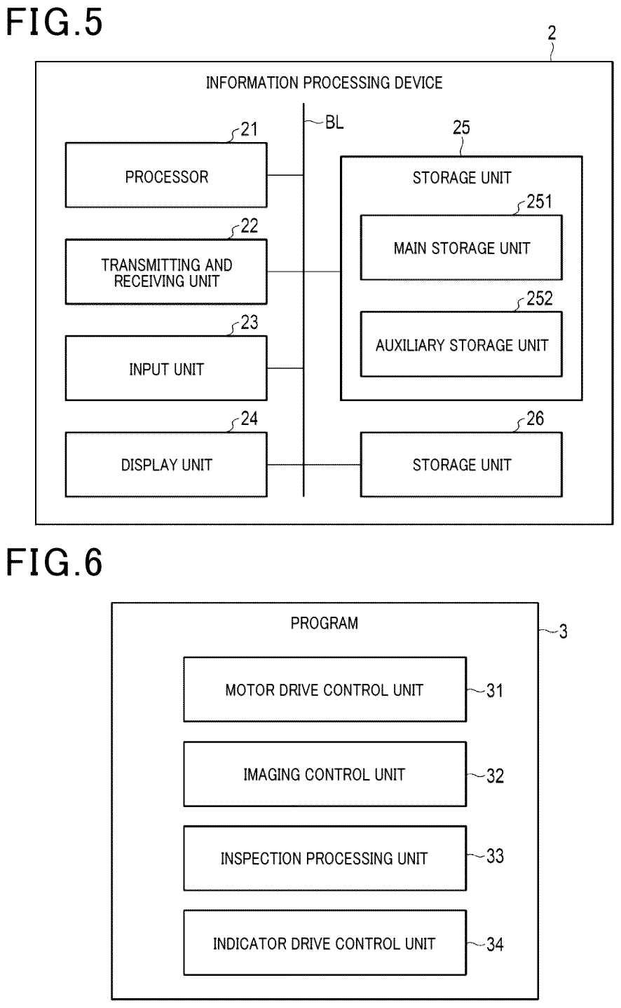

FIG. 5 is a diagram illustrating an example hardware configuration of the information processing device 2.

The information processing device 2 includes a processor 21, a transmitting and receiving unit 22, an input unit 23, a display unit 24, a storage unit 25 (main storage unit 251, auxiliary storage unit 252) and a storage unit 26.

The processor 21 is responsible for controlling the entire device, and may execute, for example, inspection programs stored in the main storage unit 251 and perform various processing using the auxiliary storage unit 252 as the work area.

The transmitting and receiving unit 22 corresponds to a communication device that transmits and receives information to and from the inspection device 1.

The input unit 23 corresponds to an input device such as a pointing device that accepts information input by the user, and may be realized by using, for example, a tablet. The display unit 24 corresponds to a display device that displays the inspection results and the final sorting results.

The storage unit 25 (main storage unit 251, auxiliary storage unit 252) corresponds to a memory such as a read only memory (ROM) and a random access memory (RAM) that stores various information.

The storage unit 26 corresponds to a storage device made of a magnetic material or a semiconductor, and accumulates multiple images obtained from the inspection device 1 and the weight data of the object to be inspected.

FIG. 6 is a diagram illustrating an example functional configuration of an inspection program executed by the processor 21.

An inspection program 3 executed by the processor 21 has, as various functions, a motor drive control unit 31, an imaging control unit 32, an inspection processing unit 33 and an indicator drive control unit 34.

### Motor Drive Control Unit

The motor drive control unit 31 controls the output of the motor 15 which serves as a power source for the gears 15a that drive the rotation table 11. Since the rotation table 11 is usually driven at a constant speed, the motor drive control unit 31 performs only the on/off control. However, the motor drive control unit 31 may change the rotation speed of the rotation table 11 by controlling the output of the motor 15 depending on the processing speed of each unit (such as imaging speed) and the work efficiency.

### Imaging Control Unit 32

The imaging control unit 32 controls the shutter timing of the imaging device 12.

The imaging control unit 32 transmits a shutter instruction signal to the inspection device in accordance with the movement of the rotation table 11 and the timing of the weight measurement and operates the shutter of the imaging device 12. Since the rotation table 11 is usually driven at a constant speed, the imaging control unit 32 controls the shutter timing to be constant. However, the imaging control unit 32 can change the shutter timing depending on the processing speed of each unit (such as imaging speed) and the work efficiency.

### Inspection Processing Unit

The inspection processing unit 33 performs inspections for various inspection criteria using the images of the object to be inspected acquired by imaging and the weight data of the object to be inspected. The inspection processing unit 33 uses image processing, artificial intelligence, and the like to obtain the final inspection results (such as ranks) and display them on the display unit 24.

### Indicator Drive Control Unit 34

The indicator drive control unit 34 controls the indicator 16 of the inspection device 1, and may change the color of the indicator 16, for example, depending on the processing status of the inspection criteria.

### Operation of Inspection System

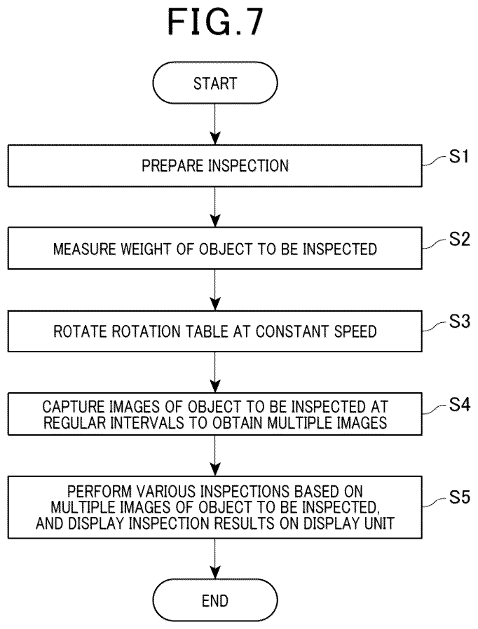

FIG. 7 is a flowchart showing an overview of the operation of the inspection system.

First, preparation for the inspection is performed (S1). In this preparation, the inspection device 1 and the information processing device 2 are connected via the cable C, and the power supply of each device is turned on.

An object to be inspected is placed on the rotation table 11 of the inspection device 1.

Next, the weight of the object to be inspected is measured (S2). The weight measurement of the object to be inspected may not necessarily be performed at this stage. For example, the weight measurement of the object to be inspected may be performed after the object to be inspected is imaged. In weight measurement of the object to be inspected, the electrical resistance value (analog data) of the strain gauge obtained from the load cell 14 is converted into a load (digital data) by the A/D circuit 14a, and this is transmitted to the interface circuit unit 13 as weight data and further transmitted to the information processing device 2 via the cable C.

Next, under the control of the information processing device 2, the interface circuit unit 13 performs processing to rotate the rotation table 11 at a constant speed via the drive device including the motor 15 and the gears 15a (S3).

Next, under the control of the information processing device 2, the imaging device 12 captures images of the object to be inspected at regular time intervals to obtain multiple images (S4). The images generated by the imaging device 12 are transmitted to the information processing device 2 via the cable C.

Finally, the information processing device 2 performs inspections for various inspection criteria based on the images of the object to be inspected and the weight data of the object to be inspected, and displays the inspection results on the display unit 24 (S5).

### Details of Processing for Each of Inspection Criteria

The information processing device 2 performs inspections for various inspection criteria using the images of the object to be inspected acquired by imaging the object to be inspected at regular time intervals and the “weight” data of the object to be inspected. As described above, the inspection criteria include “size,” “deformity,” “volume,” “density,” “sugar content” and “ripeness.”

Among these inspection criteria, the technical significance of determining “volume” is particularly of importance since once “volume” is determined from the images obtained by imaging, “density” can be determined from the “volume,” and furthermore, “sugar content” can further be determined from the “density.” Therefore, a basic method for determining “volume” will be first described.

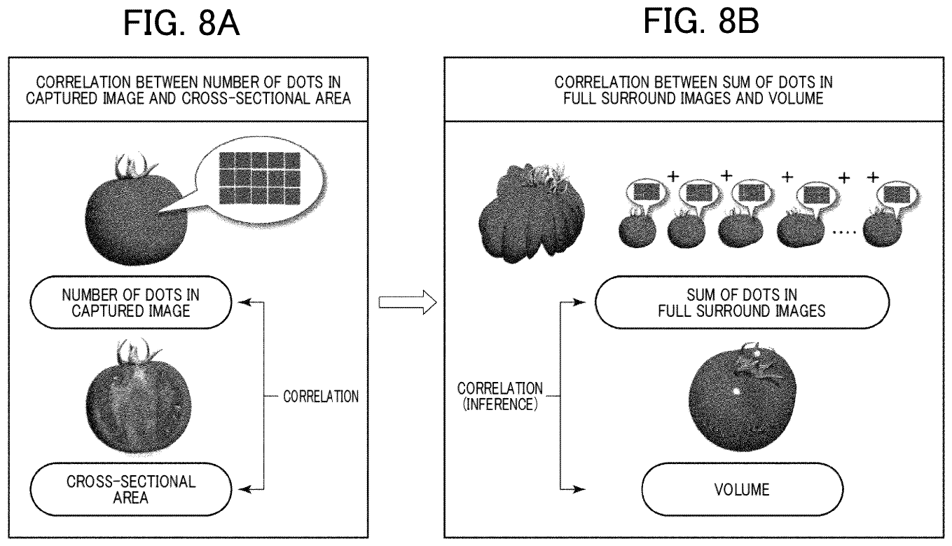

FIGS. 8A and 8B are a diagrams for explaining a basic method for determining “volume” of an object to be inspected.

In an image obtained by imaging the object to be inspected in one direction, the number of dots in the portion where the object to be inspected is imaged is correlated with the “cross-sectional area” (hereinafter, “area”) of the object to be inspected. FIG. 8A shows this correlation.

Focusing on this point, it can be inferred that, when the object to be inspected is imaged not in one direction but at regular intervals while gradually shifting the angle, the sum of the dots in the portion where the object to be inspected is imaged in each of the images (full surround images) is correlated with the “volume” of the object to be inspected. FIG. 8B shows this correlation.

Thus, the “volume” of the object to be inspected can be determined from the images of the object to be inspected obtained by imaging the object to be inspected at regular time intervals. Furthermore, from the images, the degree of “deformity” of the object to be inspected can also be determines as described below. The deformity refers to the presence of depressions or protrusions relative to the general shape of fruit and vegetables. The deformity is also called a shape defect.

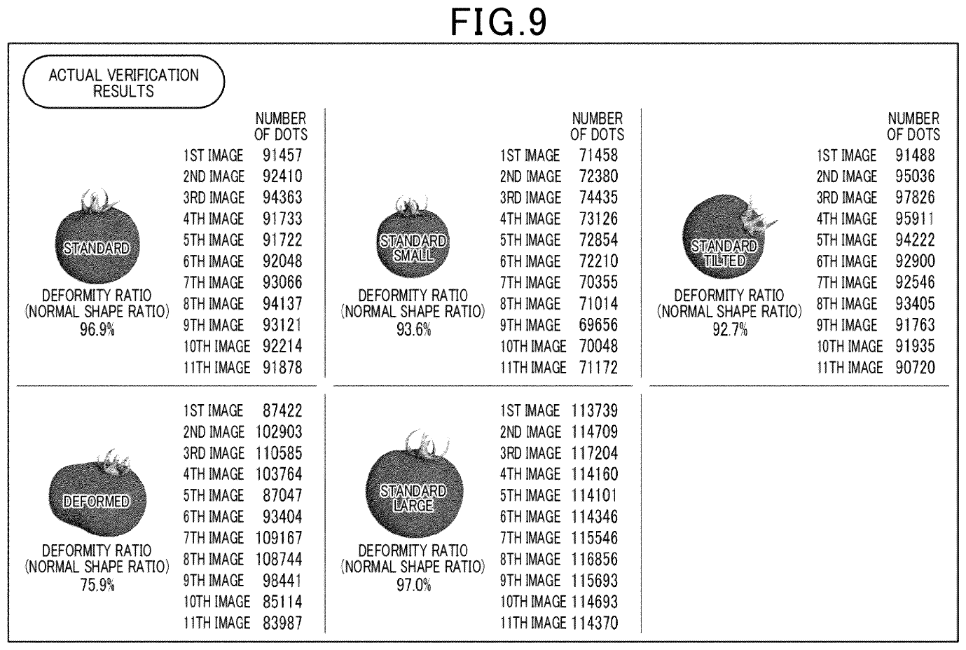

FIG. 9 is a diagram for explaining a basic method for determining “deformity” of an object to be inspected.

As described above, when the object to be inspected is imaged not in one direction but at regular intervals while gradually shifting the angle, the number of dots in the portion where the object to be inspected is imaged in each of the images (full surround images) differs to some extent among the images. Therefore, by analyzing the difference in the number of dots in the portion of each image where the object to be inspected is imaged, the degree of deformity of the object to be inspected can be determined.

FIG. 9 shows an example of actual deformity verification results for multiple objects to be inspected.

Here, 11 images were acquired as full surround images for each object to be inspected, and the ratio of the largest to smallest number of dots in each image was calculated to quantify the degree of deformity.

In the example of FIG. 9, the degree of deformity is quantified for each of five objects to be inspected having different shapes and sizes.

For the “standard” object to be inspected, the number of dots in the first image “91,457” is the smallest, and the number of dots in the third image “94,363” is the largest. Based on the ratio of the number of dots, the deformity ratio (normal shape ratio) is determined to be 96.9%.

For the “standard small” object to be inspected, the number of dots in the ninth image “69,656” is the smallest, and the number of dots in the third image “74,435” is the largest. Based on the ratio of the number of dots, the deformity ratio (normal shape ratio) is determined to be 93.6%.

For the “standard tilted” object to be inspected, the number of dots in the eleventh image “90,720” is the smallest, and the number of dots in the third image “97,826” is the largest. Based on the ratio of the number of dots, the deformity ratio (normal shape ratio) is determined to be 92.7%. The ratio is slightly smaller than the above two types of objects to be inspected, indicating a slight irregularity in shape.

For the “deformed” object to be inspected, the number of dots in the eleventh image “83,987” is the smallest, and the number of dots in the third image “110,585” is the largest. Based on the ratio of the number of dots, the deformity ratio (normal shape ratio) is determined to be 75.9%. The ratio is significantly smaller than the above three types of objects to be inspected, indicating a greater tendency of deformity.

For the “standard large” object to be inspected, the number of dots in the first image “113,739” is the smallest, and the number of dots in the third image “117,204” is the largest. Based on the ratio of the number of dots, the deformity ratio (normal shape ratio) is determined to be 97.0%. The ratio is the largest compared with the above four types of objects to be inspected, indicating a normal shape.

The following description will be given of specific methods for determining each of the inspection criteria, “size,” “deformity,” “volume,” “density,” “sugar content” and “ripeness.”

### Size

The size is determined based on one or more captured images.

First, the dimensions of the object to be inspected in the image are calculated in terms of the number of pixels. Calculation of the number of pixels requires preprocessing such as edge detection, binarization, contrast adjustment, and the like, but the description thereof will be omitted here. Any method can be used to calculate the number of pixels.

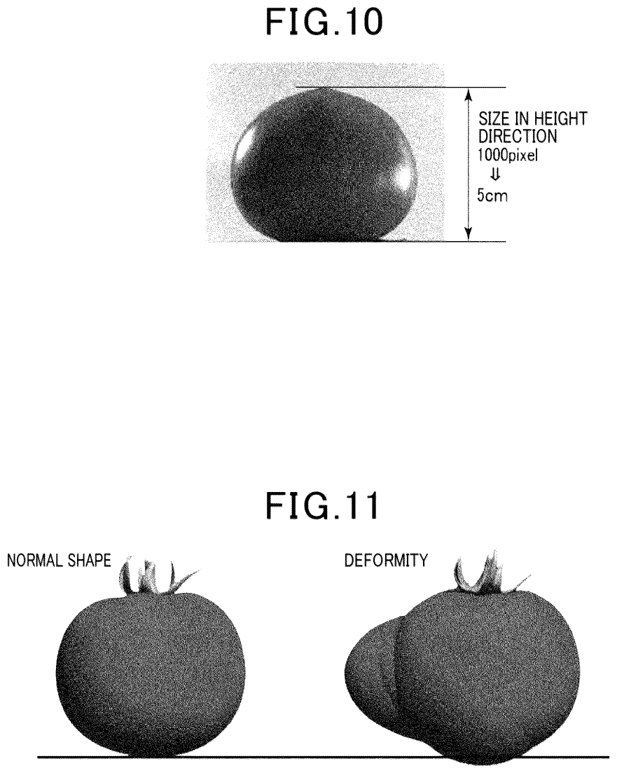

The calculated number of pixels is then converted into the actual size. For example, as shown in FIG. 10, when the number of pixels in the height direction of the object to be inspected captured in the image is 1,000 [pixel], it is converted into 5 [cm]. If the angle of view is constant, a size can be calculated by multiplying it by a predetermined constant. If it is difficult to fix the angle of view, the ratio appropriate for each angle of view may be determined, and an adjusted size may be calculated.

The size inspection result may be, for example, an average of the sizes calculated from the images. Further, the size may be expressed as a rank (grade).

### Deformity

The basic method for determining “deformity” of an object to be inspected is as described above.

The deformity is determined based on the images captured. In this example, multiple images are captured at regular intervals while rotating the object to be inspected.

For example, if there is a protrusion as shown on the right side of FIG. 11 compared with a normal shape shown on the left side of FIG. 11, the area of the object to be inspected obtained from a single captured image is larger than the area of the normal shape. Conversely, if there is a depression, the value is smaller. Therefore, by comparing the values of the area of the object to be inspected in the images captured, deformities can be detected based on (1) the ratio between the largest and smallest values of the area of the object to be inspected in each image, or (2) the degree of outliers relative to the average value.

The inspection results for deformity may be quantified as the degree of deformity (deformity ratio or normal shape ratio) based on, for example, the ratio of the largest to smallest number of dots in each image or the degree of outliers relative to the average value of the number of dots in each image. Further, the deformity may be expressed as a rank (grade).

### Volume

The basic method for determining “volume” of an object to be inspected is as described above.

The volume is determined based on the images captured. In this example, multiple images are captured at regular intervals while rotating the object to be inspected. In this example, the volume is determined based on a correlation equation between the total value of the “area” of the object to be inspected in each image and the “volume.”

The following shows the processes (A), (B) and (C) for determining the volume.

### (A) Determination of Number of Times of Imaging (Number of Images)

The number of times the object to be inspected is imaged is determined. The greater the number of times of imaging per rotation, the greater the number of images and the greater the accuracy of volume determination.

### (B) Preparation of Correlation Equation

When preparing a correlation equation between the total value of the “area” of the object to be inspected and the “volume” (hereinafter, referred to as a “volume calculation equation”), first, “reference models” for multiple sizes that approximate the shape of the object to be inspected whose volume is to be finally measured are prepared.

The reference models prepared here are not necessarily objects to be inspected whose volume is to be finally measured. For example, when it is desired to finally measure the volume of a tomato, the images prepared here may have any spherical shape similar to that of a tomato. The following shows the example in which spheres with a radius of 50 to 100 mm in 10 mm increments are prepared. In this example, six spheres with a radius r=50, 60, 70, 80, 90, 100 mm are prepared. In order to further improve the accuracy of volume determination, it is desired that the shape and size are close to those of the object to be inspected whose volume is to be finally measured. The more sizes prepared, the more accurate the approximation curve.

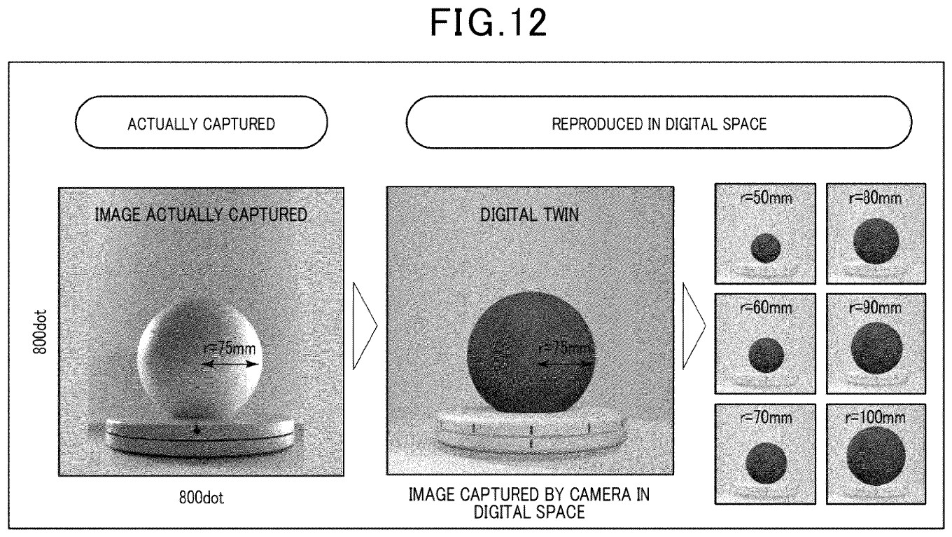

The reference model of each size is imaged for the number of images determined in the process (A) above. The left side of FIG. 12 is an example of the image obtained by actually imaging one reference model. This reference model may be reproduced as a digital twin in digital space as shown on the right side of FIG. 12.

Next, the area of the reference model is calculated. The area is calculated by counting the number of pixels (dots) that indicates the area of the object to be inspected remaining after the background is removed by image processing. This operation is performed on all the images captured, and the total number of pixels (sum of dots) is calculated.

If the reference model is defined as a perfect sphere with a radius r, rather than an irregular object, the total value of each area can be obtained by calculating “area of a perfect circle (=πr²)×number of times of imaging.”

Further, the actual volume of the reference model is determined. Although an irregular shape is measured by any method, when the reference model is a perfect sphere, the volume (=4/3 πr³) can be calculated using a calculation equation.

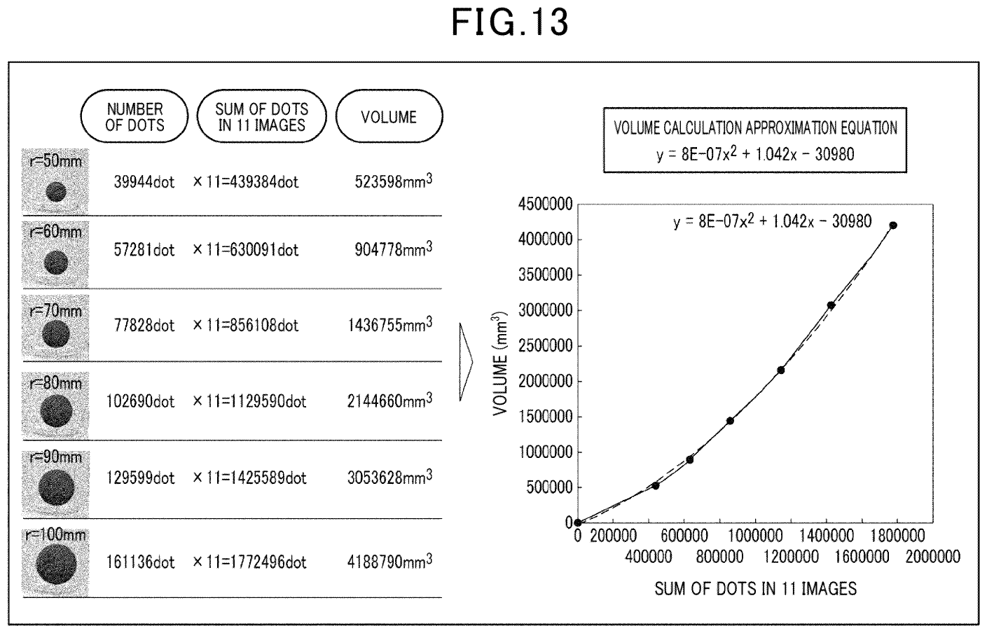

As a result of these processes, as shown on the left side of FIG. 13, the total number of pixels (sum of dots) of the images of the reference model of each size (radius r =50, 60, 70, 80, 90, 100 mm) and the volume are determined.

Next, as shown on the right side of FIG. 13, a volume calculation equation is obtained that correlates the total number of pixels already calculated with the actual volume. For example, the total number of pixels for each size is plotted against the volume. Next, an approximation equation is derived by the least squares method based on all the plotted points. The approximation function of the least squares method is not limited to a linear function, but may be an appropriate nth-order function.

The volume calculation equation may be expressed as, for example, y=8E-07x2 +1.042x-30980 (where 8E-07:0.0000008) when x is the total number of dots in the area of the reference model in each image. However, this is merely an example, and the invention is not limited thereto.

### (C) Determination of Volume

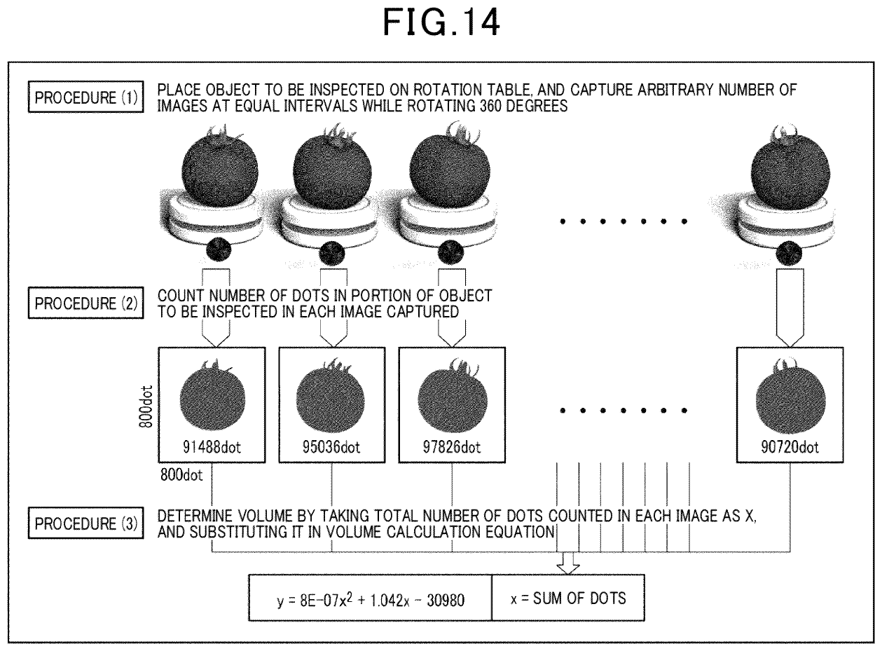

FIG. 14 shows a specific method for determining “volume” of an object to be inspected.

First, as shown in the process (1) of FIG. 14, an object to be inspected is placed on the rotation table 11, and an arbitrary number of images is captured at equal intervals while rotating it 360 degrees. The number of images is as determined in the above process

(A), and the size of the object to be inspected is close to the size of the reference model prepared in the above process (B).

Next, as shown in the process (2) of FIG. 14, the number of dots in the area of the object to be inspected in each image is counted.

Next, as shown in the process (3) of FIG. 14, the volume is determined by taking the total number of dots counted in each image as x, and substituting it for x in the volume calculation equation obtained in the above (2) (for example, y=8E-07x2+1.042x-30980). The volume may be expressed as a rank (grade).

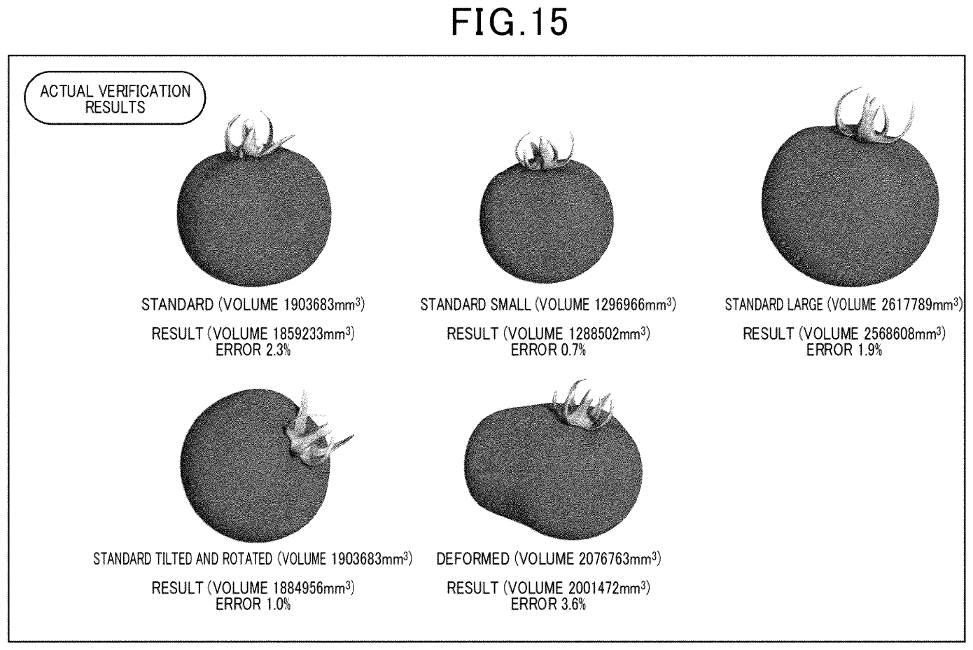

FIG. 15 shows an example of actual deformity verification results for multiple objects to be inspected.

The example of FIG. 15 shows the results of calculating the volumes of five types of tomatoes, i.e., “standard,” “standard small,” “standard large,” “standard tilted and rotated” (standard tomato rotated while placed at an angle) and “deformed,” whose volumes were known in advance.

For the objects other than “deformed,” the error from the actual volume was 2.3% or less, indicating that highly accurate inspection result was obtained. For the object “deformed,” the error was 3.6%, indicating that relatively accurate inspection result was obtained.

### Density

The density (g/cm3) of the object to be inspected is obtained by calculating “weight/volume” using the “weight” and “volume” of the object to be inspected already calculated. The density may be expressed as a rank (grade).

### Sugar Content

Sugar content is correlated with specific gravity and density. Therefore, if the specific gravity or density of the object to be inspected is known, the sugar content can be determined.

Methods for determining sugar content are complicated. Therefore, in the present embodiment, the sugar content is determined by substituting a density calculated from the volume already determined, into a correlation equation between density and sugar content, which is prepared in advance.

The sugar content inspection result may be, for example, an average of the sugar contents calculated from the images. Further, the sugar content may be expressed as a rank (grade).

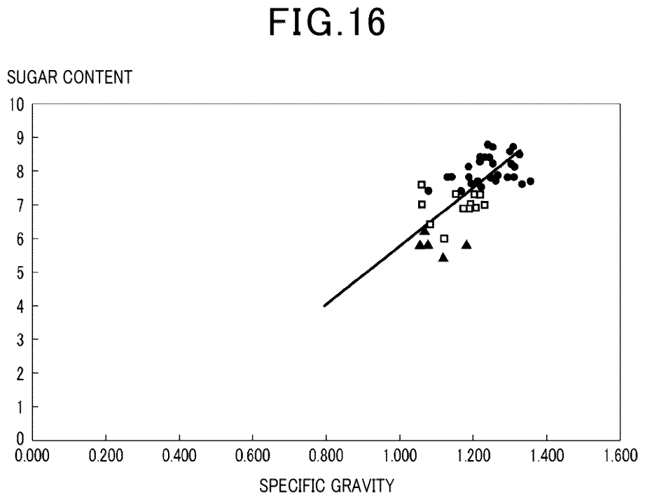

The correlation equation is prepared by measuring the sugar content of the object to be inspected in advance. The correlation equation may be a correlation equation between specific gravity and sugar content. Specific gravity is calculated from density. FIG. 16 shows the correlation between specific gravity and sugar content. Three types of symbols in FIG. 16 each represent the data of different objects to be inspected. The correlation equation is prepared based on these data.

### Ripeness

Ripeness refers to the degree of ripeness of fruits and the like, and in general, ripe and unripe fruits show differences in surface color. Examples include tomatoes and apples. Such fruits change the surface color from green to red as they ripen.

In the present embodiment, in which the outer peripheral surface of the object to be inspected is imaged, it is possible to quantify the change in surface color using multiple images obtained by imaging. Specifically, the ripeness is determined by calculating “the number of pixels of a color that appears when the fruit is ripe (red for tomatoes and apples)” /“the total number of dots in the measured outer perimeter.” The ripeness may be expressed as a rank (grade).

### Application Examples

The inspection system can be applied in various ways as described below.

### Application of Multiple Inspection Devices

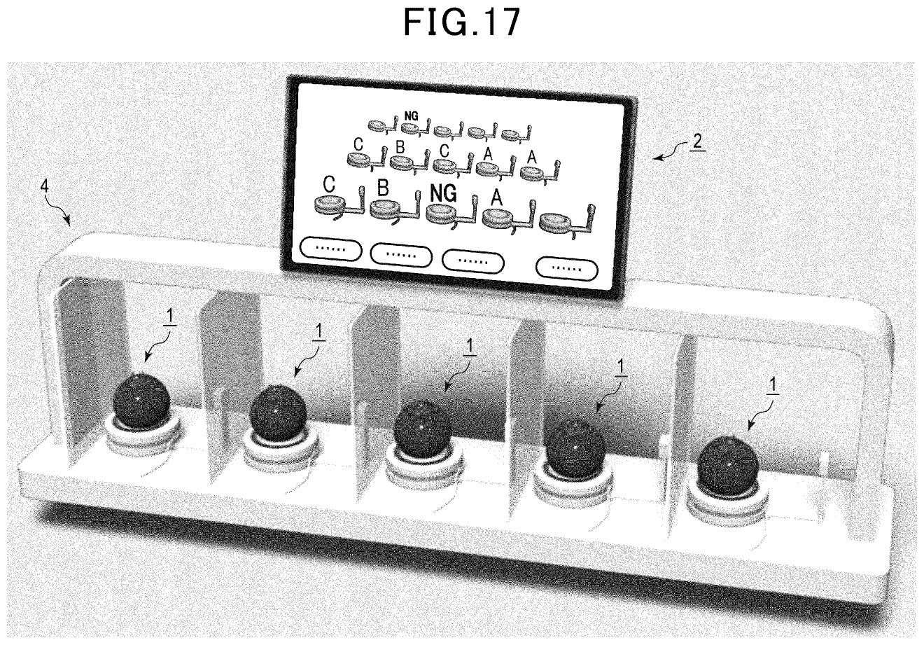

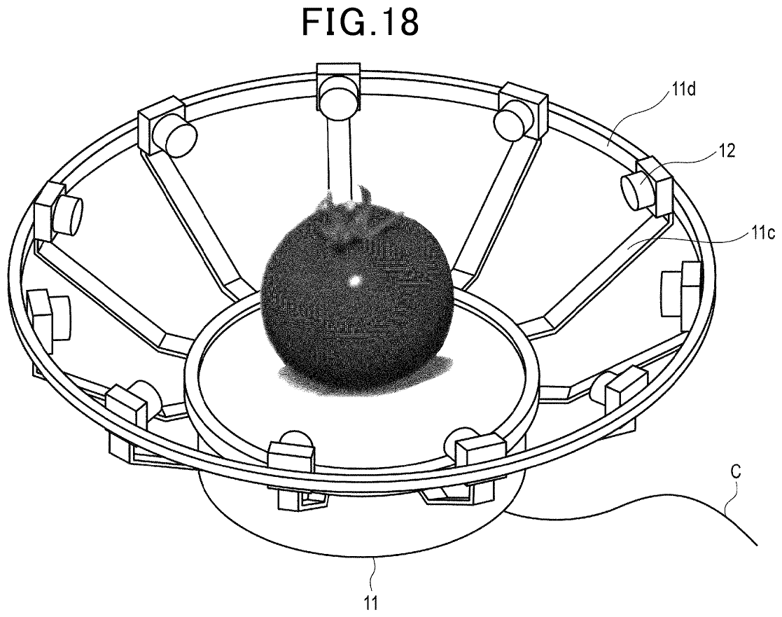

FIG. 17 is a diagram illustrating an example in which multiple devices identical to the above-mentioned inspection device 1 are provided and arranged on an inspection table 4 for collectively performing inspection.

As shown in FIG. 17, the multiple inspection devices 1 are disposed on the inspection table 4, together with an information processing device 2 having a display unit. The multiple inspection devices 1 are connected to the information processing device 2 via the above-mentioned cable C or wireless communication. The multiple inspection devices 1 are controlled by the information processing device 2 so that the results of each inspection are displayed on the information processing device 2.

By increasing the number of the rotation tables 11 as described above, the processing capability per unit time can increase. Further, by aligning the imaging direction with the direction in which the inspection objects are arranged, a partition can be used as the background. This enables stable background processing by image processing or the like.

Application to AI Visual Inspection using Captured Images

Artificial intelligence (AI) may be used for visual inspection of objects to be inspected. Visual inspection using AI requires a learning model to be prepared in advance, which usually requires a large number of images. Therefore, images of the object to be inspected captured with the inspection device 1 can be accumulated and used for AI learning to improve the accuracy of visual inspection by AI. In this case, the inspection device 1 also functions as an imaging device for obtaining images required for AI learning.

As described above in detail, the embodiment enables easy inspection of objects to be inspected such as fruit and vegetables with a simple configuration.

In this embodiment, the rotation speed of the rotation table 11 is constant, but the rotation speed of the rotation table 11 does not necessarily have to be constant. Further, the time interval of imaging with the imaging device 12 does not necessarily have to be constant. However, the positions of imaging are matched with the imaging device 12 when imaging the object to be inspected and when imaging the reference model. Therefore, it is desired that the rotation speed of the rotation table 11 and the time interval of imaging with the imaging device 12 be the same when imaging the object to be inspected and when imaging the reference model. Even if the positions of imaging with the imaging device 12 do not match when imaging the object to be inspected and when imaging the reference model, the volume can be correctly calculated using the above-mentioned volume calculation equation if the object to be inspected is close to a perfect sphere, such as a tomato or melon.

### Modified Examples of Inspection Device

The following description will be given of some modified examples of the inspection device 1.

### Modified Example 1

In the aforementioned embodiment, a single imaging device 12 is provided. In Modified Example 1, two or more imaging devices 12 may be provided. In this case, two or more supports 12a are provided around the outer periphery of the case 10 such that each of the imaging devices 12 is supported by each of the supports 12a. The two or more supports 12a may be equally or unequally spaced from each other. Due to the two or more imaging devices 12 being provided, it is possible to capture two or more images of the object to be inspected by single imaging with each imaging device 12. This reduces the number of times of imaging required to obtain the number of images of the object to be inspected needed to determine the volume.

Further, increasing the number of imaging devices 12 can reduce the amount of rotation of the rotation table 11 required to obtain the number of images of the object to be inspected needed to determine the volume. That is, it is not necessary to perform imaging while rotating the rotation table 11 as long as a sufficient number of imaging devices 12 are used for imaging.

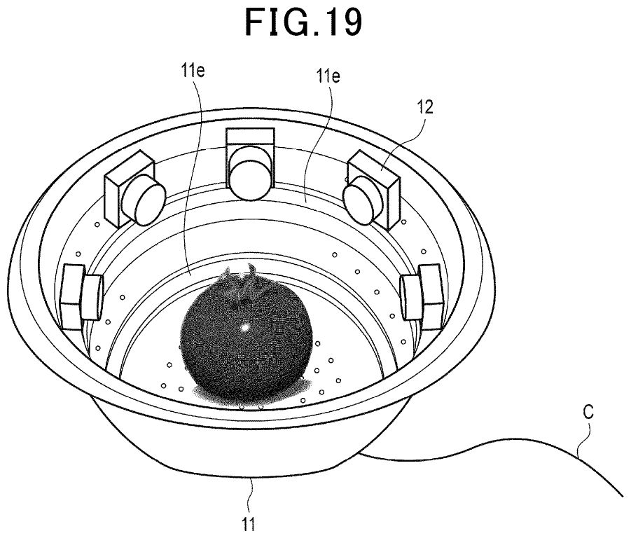

FIG. 18 is a diagram illustrating an appearance of an inspection device according to a first example of Modified Example 1. In the first example, multiple supports 11c are disposed around the non-rotatable base 11. The supports 11c are bent obliquely upward from the outer periphery of the base 11 with their distal ends at a constant height from the base 11. The distal ends of the supports 11c support a support 11d having a diameter larger than that of the base 11. For example, when the size of the object to be inspected is 5 cm to 10 cm, the diameter of the base 11 is approximately 10 cm and the diameter of the support 11d is approximately 20 cm. The imaging devices 12 are fixed to the support 11d. The orientation of each imaging device 12 is adjusted so that the object to be inspected is within the angle of view of the camera module. Depending on the orientation of the imaging devices 12, images of other imaging devices 12 may appear in the image of the object to be inspected. Such images of other imaging devices 12 are removed during image processing in inspection. The removal of such images of the imaging devices 12 may be performed by any method such as pattern matching, machine learning, or the like.

In FIG. 18, the imaging devices 12 are fixed at the intersections between the supports 11c and the support 11d. However, the imaging devices 12 are not necessarily fixed at the intersections between the supports 11c and the support 11d. Further, the number of supports 11c and the number of imaging device 12 are not necessarily the same. Moreover, in FIG. 18, the supports 11c are equally spaced around the outer periphery of the base 11. However, the supports 11c may be unequally spaced around the outer periphery of the base 11. In addition, the fixed positions of the imaging devices 12 may also be unequally spaced.

The height of the distal end from the base 11 may be such that, for example, when an object to be inspected is placed on the base 11, the optical axis of the camera module of the imaging device 12 coincides with the center of the object to be inspected. In practice, the size of the fruit and vegetables as the objects to be inspected is not uniform, so the height of the distal end from the base 11 may be defined by the above-mentioned reference model. Furthermore, in FIG. 18, the imaging devices 12 are fixed to the support 11d. However, the imaging devices 12 may be fixed to the supports 11c, and in this case, the support 11d may be omitted. The heights of the imaging devices 12 supported by the base 11 may be different.

FIG. 19 is a diagram illustrating an appearance of an inspection device according to a second example of Modified Example 1. The structure of the first example is a skeleton structure formed of the supports 11c and the support 11d. On the other hand, the structure of the second example is a continuous structure bent obliquely upward from the base 11. In the second example, the base 11 may be formed as, for example, a cage body having a living hinge 11e. An object to be inspected is placed on the bottom of the base 11 of the cage body. Further, the imaging devices 12 are disposed on a shoulder formed by bending the living hinge 11e. The orientation of each imaging device 12 is adjusted so that the object to be inspected is within the angle of view of the camera module. In the second example, the installation height of the imaging devices 12 can be changed by changing the bending position of the living hinge 11e, that is, the position where the shoulder is formed.

In FIG. 19, the imaging devices 12 may not necessarily be equally spaced in the circumferential direction of the base 11 but may be unequally spaced. Further, the imaging devices 12 can be installed at different heights from the base 11 by forming multiple shoulders of different heights by bending the living hinge 11e multiple times.

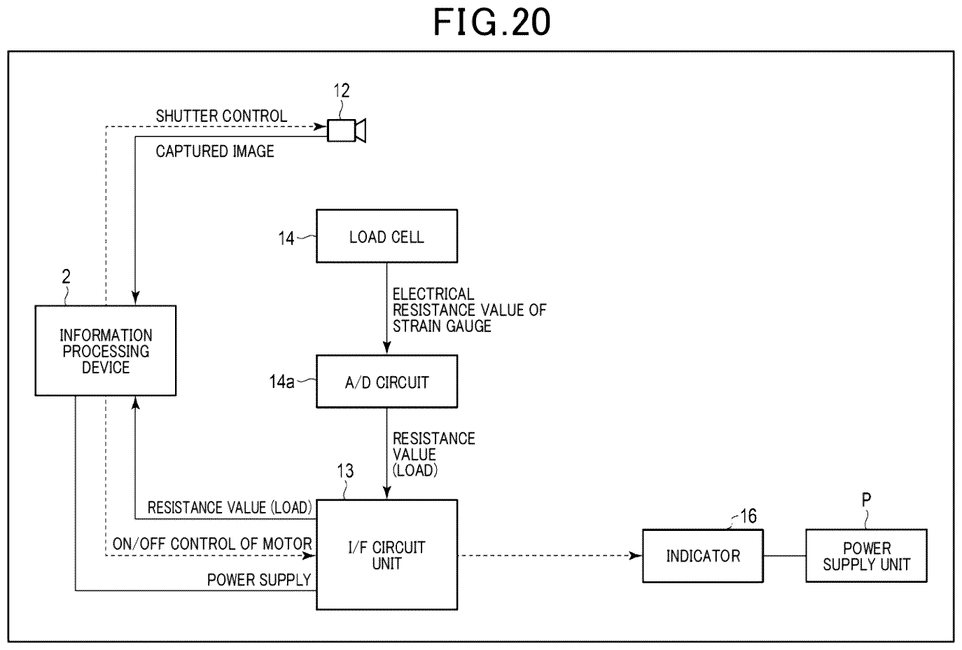

FIG. 20 is a block diagram illustrating a configuration of the inspection device according to Modified Example 1. The configuration of FIG. 20 can be applied to both the first and second examples. In Modified Example 1, the inspection device 1 includes an interface circuit unit 13, a load cell (weight measuring device) 14, an A/D circuit 14a, a power supply unit (including a battery) P, an indicator 16, and the like. That is, in Modified Example 1, since the base 11 does not need to be rotated, a motor 15 and gears 15a are not necessary. Further, in Modified Example 1, the case 10 and the base 11 shown in FIG. 2 may be taken as a single base 11. In this case, the interface circuit unit 13, the load cell 14, the A/D circuit 14a, the power supply unit P and the indicator 16 can all be mounted in the base 11.

The configurations of the interface circuit unit 13, the load cell 14, the A/D circuit 14a, the power supply unit P and the indicator 16 are the same as those described with reference to FIG. 4. Therefore, the description will be omitted. Further, the configuration of the information processing device 2 is also basically the same as that described with reference to FIG. 5. However, the inspection program 3 executed by the processor 21 does not necessarily function as the motor drive control unit 31.

Further, the inspection performed by the inspection processing unit 33 is also basically the same as that described with reference to FIGS. 7 to 14. However, in Modified Example 1, the operation of S3 in FIG. 7 is not necessary. Further, in Modified Example 1, the number of times of imaging used for calculating the volume is the number of the imaging devices 12 mounted on the base 11.

As described above, Modified Example 1 does not require a mechanism for rotating the object to be inspected. Therefore, easy inspection of objects to be inspected such as fruit and vegetables can be performed with a simple configuration.

### Modified Example 2

In the aforementioned embodiment, the rotation table 11 rotates, and the object to be inspected is rotated with the rotation of the rotation table 11 and is imaged by the imaging device 12, whereby the full surround images of the object to be inspected can be obtained. Alternatively, full surround images of the object to be inspected can be obtained by fixing the object to be inspected and imaging the object to be inspected by the imaging device 12 rotating around the object to be inspected.

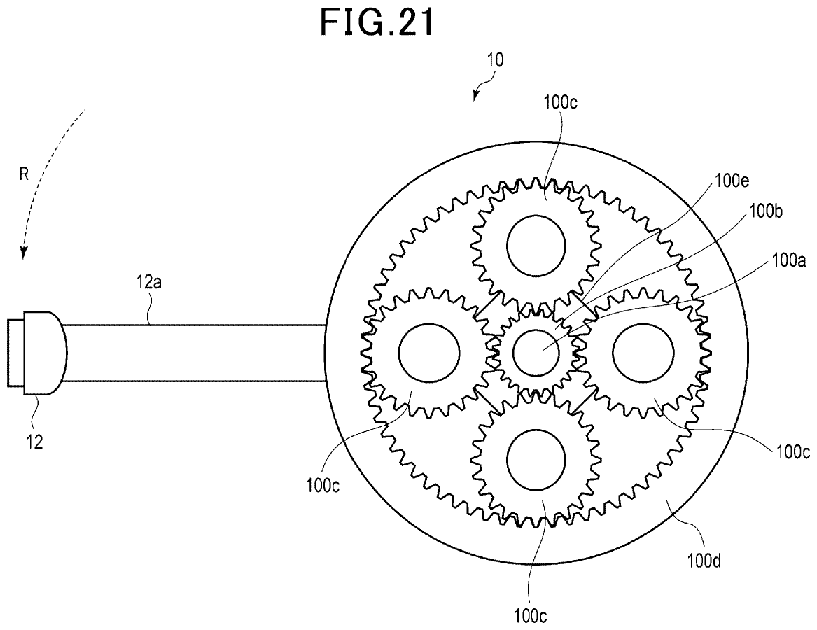

FIG. 21 is a diagram illustrating an internal configuration of the case 10 in Modified Example 2. In Modified Example 2, a drive device including gears is provided in the case 10. The drive device includes a planetary gear mechanism. The planetary gear mechanism includes a shaft 100a, a sun gear 100b, planetary gears 100c, an internal gear 100d and a planetary carrier 100e.

The shaft 100a is a stationary shaft around which the sun gear 100b is fitted. The shaft 100a is attached to the base 11, and also supports the base 11.

The sun gear 100b meshes with four planetary gears 100c arranged around it and is fixed to the shaft 100a.

Each of the planetary gears 100c meshes with the sun gear 100b and also meshes with the internal gear 100d. The planetary gears 100c are connected to each other by the planetary carrier 100e. The planetary carrier 100e can be rotated by the motor 15.

The internal gear 100d forms at least part of the outer periphery of the case 10. Further, the support 12a is attached to the outer periphery of the case 10. The support 12a supports the imaging device 12.

In the configuration shown in FIG. 21, the planetary carrier 100e is rotated by the motor 15, causing the four planetary gears 100c connected by the planetary carrier 100e to rotate while revolving around the sun gear 100b. Due to the four planetary gears 100c revolving, the internal gear 100d rotates, for example, in the R direction shown in the figure, and the case 10 rotates accordingly. Since the support 12a is attached to the outer periphery of the case 10, the rotation of the case 10 causes the support 12a to rotate, for example, in the R direction.

Accordingly, the imaging device 12 supported by the support 12a also rotates, for example, in the R direction. Meanwhile, the shaft 100a does not rotate, so the base 11 supported by the shaft 100a does not rotate either. Therefore, the imaging device 12 can rotate relatively to the object to be inspected placed on the base 11. When the object to be inspected is imaged by the imaging device 12 described above, the same full surround images of the object to be inspected as those described in the embodiment above can be obtained. In Modified Example 2, the rotation speed of the internal gear 100d does not necessarily have to be constant. Further, the time interval of imaging with the imaging device 12 does not necessarily have to be constant.

The case 10 is provided with the cable C. In Modified Example 2, since the support 12a is rotated by the rotation of the case 10, the support 12a while rotating may come into contact with the cable C. In order to avoid such contact between the support 12a and the cable C while the support 12a is rotating, it is desired that the installation height of the support 12a to the case 10 is determined taking into consideration the flexure or the like of the cable C. Alternatively, the case 10 may be divided into a rotatable portion to which the internal gear 10d is attached and a non-rotatable portion. In this case, the cable C and the like may be provided on the non-rotatable portion of the case 10.

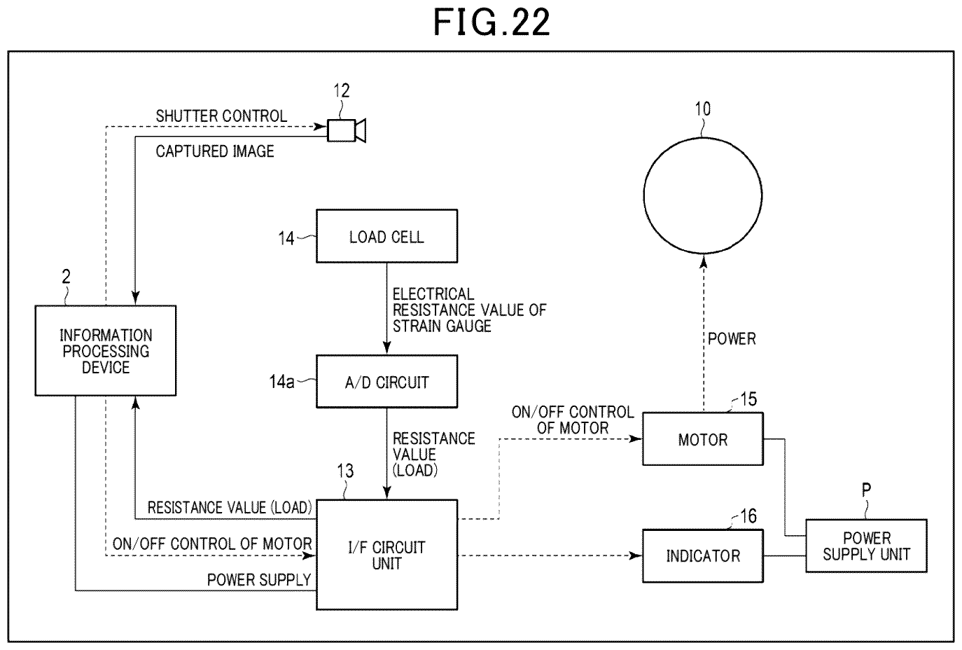

FIG. 22 is a block diagram illustrating a configuration of an inspection device according to Modified Example 2. In Modified Example 2, the inspection device 1 includes an interface circuit unit 13, a load cell (weight measuring device) 14, an A/D circuit 14a, a power supply unit (including a battery) P, an indicator 16, a motor 15, and the like. Unlike FIG. 4, the motor 15 in FIG. 22 is configured to rotate the case 10, more specifically the planetary carrier 10e. The other configurations are the same as those in FIG. 4.

Further, the inspection performed by the inspection processing unit 33 is also basically the same as that described with reference to FIGS. 7 to 14. However, in Modified Example 2, the case 10 is rotated instead of the rotation table 11 in S3 of FIG. 7.

As described above, Modified Example 2 enables easy inspection of objects to be inspected such as fruit and vegetables with a simple configuration. Further, in Modified Example 2, the base on which an object to be inspected is placed does not rotate. This makes it easier to stabilize the position of the object to be inspected.

The present invention is not limited to the embodiments as described above, and the components can be modified and embodied when implemented, without departing from the spirit of the invention. Further, multiple components disclosed in the above embodiments can be appropriately combined to form various inventions. For example, some components may be removed from all the components shown in the above embodiments. Furthermore, components from different embodiments can be appropriately combined.

In recent years, against the backdrop of labor shortages in agriculture, automation of inspection and sorting of fruit and vegetables is being promoted. Current fruit sorting criteria include criteria that are easy to measure and quantify (weight and length), as well as criteria that evaluate internal quality difficult to measure (sugar content and acidity), and criteria that evaluate external quality whose evaluation indicators are complex (rot and blemishes). The entire contents of these publications are incorporated herein by reference.

Introducing automated devices instead of manual inspection and fruit sorting improves processing capability, but the system becomes more complex, requiring huge equipment costs and a large installation space, and also becomes difficult to handle.

An inspection device and an inspection method according to embodiments of the present invention enable easy inspection of objects to be inspected such as fruit and vegetables with a simple configuration.

An inspection device according to one aspect of the present invention includes: a base on which an object to be inspected is placed; a drive device that rotates the base on which the object to be inspected is placed; and an imaging device that images the object to be inspected.

According to an embodiment of the present invention, easy inspection of objects to be inspected such as fruit and vegetables can be performed with a simple configuration.

Obviously, numerous modifications and variations of the present invention are possible in light of the above teachings. It is therefore to be understood that within the scope of the appended claims, the invention may be practiced otherwise than as specifically described herein.

1. An inspection device, comprising:
a base on which an object to be inspected is placed;
a drive device configured to rotate the base on which the object to be inspected is placed; and
an imaging device that images the object to be inspected.

2. The inspection device according to claim 1, further comprising:
an information processing device comprising circuitry configured to control the imaging device to image the object to be inspected while rotating the object to be inspected such that the circuitry of the information processing device acquires a plurality of images of the object to be inspected from the imaging device and performs inspections of the object to be inspected based on the plurality of images of the object to be inspected.

3. The inspection device according to claim 2, wherein the circuitry of the information processing device is configured to determine a volume of the object to be inspected based on a sum of predetermined information obtained from each of the images.

4. The inspection device according to claim 2, wherein the circuitry of the information processing device is configured to determine a degree of deformity of the object to be inspected based on predetermined information obtained from each of the images.

5. The inspection device according to claim 2, wherein the circuitry of the information processing device is configured to determine a size of the object to be inspected based on predetermined information obtained from each of the images.

6. The inspection device according to claim 3, further comprising:
a weight measuring device that measures a weight of the object to be inspected,
wherein the circuitry of the information processing device is configure to determine a density of the object to be inspected based on a volume and a weight of the object to be inspected.

7. The inspection device according to claim 6, wherein the circuitry of the information processing device is configured to determine a sugar content of the object to be inspected based on a density of the object to be inspected.

8. The inspection device according to claim 2, wherein the circuitry of the information processing device is configured to determine ripeness of the object to be inspected based on predetermined information obtained from each of the images.

9. The inspection device according to claim 2, wherein the base, the drive device and the imaging device are provided in a single inspection device, and the inspection device is connected to the information processing device.

10. The inspection device according to claim 9, wherein the inspection device is provided in a plurality such that the plurality of the inspection devices is connected to the information processing device.

11. The inspection device according to claim 1, further comprising:
a case configured to hold the base in a rotatable manner; and
a support attached to the case and configured to support the imaging device.

12. An inspection device, comprising:
a base on which an object to be inspected is placed;
a structure bent obliquely upward from the base; and
a plurality of imaging devices supported by the structure and configured to image the object to be inspected in different directions.

13. The inspection device according to claim 12, wherein the structure is a skeleton structure comprising a plurality of first supports bent obliquely upward from the base, and a ring-shaped second support supported by distal ends of the plurality of first supports.

14. The inspection device according to claim 12, wherein the structure is a continuous structure bent obliquely upward from the base, the structure having a living hinge.

15. An inspection device, comprising:
a base on which an object to be inspected is placed;
a case configured to support the base in a non-rotatable manner;
a drive device positioned in the case and configured to rotate the case; and
a support attached to the case and supporting an imaging device configured to image the object to be inspected.

16. The inspection device according to claim 15, wherein the drive device comprises a fixed sun gear, a plurality of planetary gears configured to mesh with the sun gear and revolve around the sun gear, a planetary carrier that connects the plurality of planetary gears and connected to a motor, and an internal gear configured to mesh with the plurality of planetary gears and rotate as the plurality of planetary gears revolve such that at least a portion of the case to which the support is attached is rotated.

17. An inspection method, comprising:
causing a drive device to rotate a base on which an object to be inspected is placed;
controlling an imaging device such that the imaging device images the object to be inspected while rotating the object to be inspected and acquiring a plurality of images of the object to be inspected from the imaging device; and
performing inspections of the object to be inspected based on the plurality of images of the object to be inspected.
